(19) United States
(12) Patent Application Publication
(10) Pub. No.: US 2025/0058095 A1
(43) Pub. Date: Feb. 20, 2025

(54) RETENTION SYSTEM AND METHOD FOR DISPOSABLE CARTRIDGE FOR ELECTROSTATIC APPLICATOR

(71) Applicant: Octet Medical, Inc., San Diego, CA (US)

(72) Inventors: Clifford A. Wright, San Diego, CA (US); Kyle Elsabee, San Diego, CA (US); Ron Lawrence, San Diego, CA (US); Michael Williams, San Diego, CA (US); Tom Lorenzana, San Diego, CA (US)

(73) Assignee: Octet Medical, Inc., San Diego, CA (US)

(21) Appl. No.: 18/235,324

(22) Filed: Aug. 17, 2023

(51) Int. Cl. A61M 35/00

(52) U.S. Cl. CPC A61M 35/003

(57) ABSTRACT

A disposable fluid delivery system for an electrostatic applicator, is disclosed. The system can include a cartridge housing to store an aqueous solution and a nozzle housing. A high voltage contact can be electrically attached to a delivery tube at least partially within the nozzle housing, the high voltage contact in electrical communication with a high voltage module and configured to electrostatically charge fluid contents within the delivery tube. A cartridge retention system can be positioned at least partially with an outer surface of the cartridge housing, the cartridge retention system configured to engage with and retain the cartridge housing in a connected configuration with a cartridge receiving chamber of an applicator housing of the electrostatic applicator.

### FIELD

The solution of this disclosure relates to retention systems for use with devices, systems, and methods for applying one or more medicaments (e.g., one or more biologics, polymer spun wound dressing, antiseptics, or anesthetic) to a treatment site (e.g., a wound surface of subject).

### BACKGROUND

Infectious disease is too often acquired in places that should be safe, such as ambulances, hospitals, clinical settings, and other areas such as assisted living facilities. Indeed, these health care associated infections (HAI) pose a major threat to patient safety and cause an unnecessary financial burden. Surgical infections, for example, which are a large contributor to HAIs, can create pain and discomfort for a patient but also can contribute to longer and/or repeat hospital admissions. Numerous antibacterial and/or analgesic compounds are available to help treat the patient and avoid infections; however, despite the prevalence of these compounds, current delivery methods can often be less than efficacious.

Oral and intravenous administration, for example, are often insufficient to effectively control severe pain at or treat a specific region of the human body and giving a high concentration dose may lead to adverse events. To overcome some of the issues related to oral and intravenous administration, delivery vehicles such as hydrogels have been developed to provide spatial and temporal control over the release of various therapeutic agents, including small molecule drugs, peptides, and cells. However, hydrogels can also have certain undesirable characteristics, including being expensive and difficult to sterilize.

More recently, electrospraying has emerged as a technology with potential biomedical applications. Electrospraying includes providing an electrostatic charge to a fluid as the fluid is expelled from an electrostatic sprayer. The electric field can cause the expelled liquid to break up into diminutive droplets, e.g., on the order of microns, which can bind to a treatment site relatively evenly and use less solution. That said, not all solutions, including antiseptics and/or analgesics, respond equally to the same electrostatic conditions-differences in the viscosity and/or dielectric properties of the solution can require different optimal configurations for an electrostatic sprayer device. Current electrostatic sprayer devices do not provide modular systems that can account for various types of different solutions within one electrostatic sprayer device. This disclosure resolves these and other issues of the art.

### SUMMARY

The subject of this disclosure is an electrostatic applicator for emitting contents from a disposable cartridge (e.g., treatment solution contained in the cartridge) to a treatment site of a patient.

In some examples, a disposable fluid delivery system for an electrostatic applicator, is disclosed. The system can include a cartridge housing to store an aqueous solution and a nozzle housing. A high voltage contact can be electrically attached to a delivery tube at least partially within the nozzle housing, the high voltage contact in electrical communication with a high voltage module and configured to electrostatically charge fluid contents within the delivery tube. A cartridge retention system can be positioned at least partially with an outer surface of the cartridge housing, the cartridge retention system configured to engage with and retain the cartridge housing in a connected configuration with a cartridge receiving chamber of an applicator housing of the electrostatic applicator.

In some examples, the cartridge retention system includes a latch with an angled face projecting from a surface of the cartridge housing (e.g., a lower surface, a side surface, or an upper surface) and configured to securely connect with an angle surface of a cam release of the cartridge receiving chamber.

In some examples, the cartridge retention system includes a pull-down lock including a ramped face and a restriction guide slidably connected to the pull-down lock.

In some examples, the pull-down lock includes a lock housing with an upper ramped catch. The ramped face is positioned on an outer edge of the lock housing adjacent or on a surface opposite the upper ramped catch.

In some examples, the restriction guide includes a planar base with a guide of the restriction guide extended upward away from the planar base. The restriction guide can be connected to a spring.

In some examples, the cartridge retention system includes one or more compressible portions of the cartridge housing that form a living hinge configured to snap into recesses.

In some examples, the cartridge retention system includes an aperture in the outer surface of the cartridge housing, the aperture arranged to receive a movable pin of a solenoid that locks the cartridge housing in a connected configuration.

In some examples, an electrostatic applicator system is disclosed for delivering a treatment solution to a target site. The system includes a portable reusable electrostatic applicator including a device housing configured to be handheld, a motor in the device, a voltage source, a high voltage module electrically connected to the voltage source, and a cartridge chamber defined with one or more outer surfaces of the device housing. A disposable cartridge is removably insertable in the cartridge chamber, the disposable cartridge including a cartridge housing configured to an aqueous solution and a nozzle housing. A high voltage contact can be electrically attached to a delivery tube at least partially within the nozzle housing. The high voltage contact can be in electrical communication with a high voltage module and electrostatically charge fluid contents within the delivery tube. A cartridge retention system can be positioned at least partially with an outer surface of the cartridge housing, the cartridge retention system configured to engage with and retain the cartridge housing in a connected configuration with a cartridge receiving chamber of an applicator housing of the electrostatic applicator.

In some examples, a cam release is extended from within the device housing through an aperture of a wall of the cartridge chamber. A motor can be within the device housing to rotate the cam release and release a latch of the cartridge retention system and cause the cartridge housing to transition to a disconnected configuration.

In some examples, upon releasing the latch, one or more bias elements cause the cartridge housing to be urged away from the cartridge chamber.

In some examples, the latch of the cartridge retention system is angled to receive an angled face of the cam release.

In some examples, a pull-down lock is configured to engage with a relief of the cartridge retention system, the pull-down lock extended from within the device housing through an aperture of a wall of the cartridge chamber, the pull-down lock including a ramped face. A restriction guide can be slidably connected to the pull-down lock, wherein a motor within the device housing is configured to rotate on the ramped face of the pull-down lock so that the motor is only capable of moving, by the restriction guide, in a vertical and/or angled direction to transition to a disconnected configuration.

In some examples, the pull-down lock includes a lock housing with an upper ramped catch extended through the aperture, and wherein the ramped face is positioned on an outer edge of the lock housing adjacent or on a surface opposite the upper ramped catch.

In some examples, the restriction guide includes a planar base with a guide of the restriction guide extended upward away from the planar base, wherein the restriction guide is connected to a spring that maintains the pull-down lock when the cartridge housing slides into the cartridge chamber thereby locking the cartridge housing in place when the relief in the cartridge housing aligns with the pull-down lock.

In some examples, the cartridge retention system includes one or more compressible portions of the cartridge housing that form a living hinge configured to snap into recesses of the cartridge chamber so as to lock the cartridge housing in the cartridge chamber.

In some examples, a solenoid including a pin extended from within the device housing through an aperture of a wall of the cartridge chamber, wherein the pin is caused to be pushed inward as the cartridge housing slides within the cartridge chamber over the pin of the solenoid until the pin is released to extend through an aperture in the cartridge retention system thereby locking the cartridge housing in a connected configuration.

In some examples, to transition the cartridge housing to a disconnected configuration, the solenoid is energized and the pin is released from the aperture in the outer surface of the cartridge housing.

In some examples, a method is disclosed for operating an electrostatic applicator system. The method can include inserting a disposable cartridge into a cartridge chamber of an electrostatic applicator system until a voltage contact of the disposable cartridge contacts a voltage contact of the electrostatic applicator system and a cartridge retention system causes a cartridge housing of the disposable cartridge to removably connect to the cartridge chamber of an applicator housing of the electrostatic applicator in a connected configuration.

In some examples, the method includes rotating, by a motor within a device housing of the electrostatic applicator, a cam release thereby releasing a latch of the cartridge retention system so that the cartridge housing transitions to a disconnected configuration.

In some examples, the method includes upon releasing the latch, one or more bias elements of the cartridge chamber cause the cartridge housing to be urged away from the cartridge chamber.

In some examples, the method includes engaging a relief of the cartridge retention system with a pull-down lock extended through an aperture in the cartridge chamber from within the device housing, the pull-down lock including a ramped face; and rotating, by a motor within the device housing, a restriction guide on and/or along a ramped face of the pull-down lock so that the motor is only capable of moving, by the restriction guide, in a vertical and/or angled direction to transition between the connected configuration and a disconnected configuration.

In some examples, the method includes releasing the cartridge housing from the connecting connected configuration to a disconnected configuration by squeezing one or more compressible portions of the cartridge housing that form a living hinge so as to unlock the cartridge housing from the cartridge chamber.

In some examples, the method includes causing, by a surface of the cartridge housing, such as a lower surface, a side surface, or an upper surface, a pin of a solenoid extended from within the device housing through an aperture of a wall of the cartridge chamber, to be pushed inward as the cartridge housing slides within the cartridge chamber until the pin is released to extend through an aperture in the cartridge retention system thereby locking the cartridge housing in the connected configuration.

In some examples, the method includes transitioning the cartridge housing to a disconnected configuration by energizing the solenoid so that the pin is released from the aperture in the outer surface of the cartridge housing.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A depicts a perspective view of an example electrostatic applicator with a disposable cartridge in an exploded state.

FIG. 1B depicts a perspective view of the example electrostatic applicator of FIG. 1A with the disposable cartridge in an assembled state.

FIG. 2A depicts a forward perspective view of the example cartridge of FIGS. 1A to 1B.

FIG. 2B depicts a rear perspective view of the example cartridge of FIGS. 1A to 1B.

FIG. 3A shows a perspective view of a cartridge receiving chamber according to exemplary aspects of this disclosure.

FIG. 3B shows a close-up of section 3B-3B of FIG. 3A.

FIG. 3C shows a lower internal perspective view of a motor operatively connected aspects of the cartridge receiving chamber of FIG. 3A according to exemplary aspects of this disclosure.

FIG. 4A shows a cross-sectional view of an exemplary cam release when coupled with aspects of a cartridge according to exemplary aspects of this disclosure.

FIG. 4B shows a lower internal perspective view of a lower surface of a cartridge according to exemplary aspects of this disclosure.

FIG. 5A shows a perspective view of a cartridge receiving chamber according to exemplary aspects of this disclosure.

FIG. 5B shows a perspective view of a cartridge when coupled with an exemplary receiving chamber according to exemplary aspects of this disclosure.

FIG. 5C shows a close-up of section 5C-5C of FIG. 5B.

FIG. 6A shows a lower internal perspective view of an exemplary motor operatively connected aspects of the cartridge receiving chamber of FIGS. 5A to 5C according to exemplary aspects of this disclosure.

FIG. 6B shows a perspective view of aspects of an exemplary motor operatively connected to aspects of the cartridge retention system of FIGS. 5A to 6A.

FIG. 6C shows a perspective view of aspects of the exemplary motor operatively connected to aspects of the cartridge retention system of FIGS. 5A to 6B.

FIG. 7 shows a side cross-section view of aspects of the exemplary motor operatively connected to aspects of the cartridge retention system of FIGS. 5A to 6C.

FIG. 8A shows a perspective view of a cartridge receiving chamber according to exemplary aspects of this disclosure.

FIG. 8B shows a perspective view of a cartridge when coupled with an exemplary receiving chamber according to exemplary aspects of this disclosure.

FIG. 9A shows a lower internal perspective view of a lower surface of the cartridge receiving chamber of FIGS. 8A to 8B according to exemplary aspects of this disclosure.

FIG. 9B shows a side cross-section view of the cartridge when coupled with the cartridge receiving chamber of FIGS. 8A to 8B according to exemplary aspects of this disclosure.

FIG. 10 shows a perspective view of an example cartridge according to exemplary aspects of this disclosure.

FIG. 11 illustrates a flowchart for a method, according to an embodiment.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In this disclosure, relative terms, such as “about,” “substantially,” or “approximately” are used to indicate a possible variation of +10% in the stated value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a treatment site of a “subject” or “patient” may be a wound site or treatment of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include, but is not limited to a doctor, surgeon, nurse, physical therapist, or other healthcare professional, or any other suitable individual, or delivery instrumentation associated with the application of a treatment solution of a treatment site of a subject.

As discussed herein, “treatment solution” may be one or more fluids (e.g., liquid and/or emulsion solution, gels, and/or mixtures) and may include one or more of an antiseptic solution, a disinfectant solution, an analgesic, an exosome, a biologic, chlorohexidine gluconate, povidone-iodine, and/or a liquid bandage solution. The analgesic can include one or more of lidocaine, levobupivacaine, acemetacin, ketorolac, and ceftazidime. The biologic can include one or more of stem cells and/or mammalian primary cells, medicaments, gels (e.g., hydrogels), reconstitute aspects (e.g., immiscible and/or lyophilized ingredients mixable with one or more solvents). The disinfectant can include one or more alcohols, aldehydes, oxidatives, phenols, quaternary ammonium compounds, etc., antibacterial agents, biguanides, analgesic agents, surfactant agents, and/or debridement components or any other contents and/or medication contemplated for storage in a cartridge of this disclosure that can be delivered (e.g., applied, deposited, and/or sprayed by an electrostatic applicator) to a treatment site of a patient. The treatment solution can include any concentration or mixture of herein disclosed ingredients.

The term “treatment solution” can also include one or more tracking materials (e.g., gels with tracking aspects, intermixed with the treatment solution). The treatment solution can include any number of small molecule drugs, peptides, cells, and other therapeutics. In some aspects, the treatment solution can include one or more active pharmaceutical ingredients, growth factors, trophic factors, exosomes, mammalian regenerative cells, and/or a supportive matrix. In some aspects, the treatment solution can include lidocaine, levobupivacaine, acemetacin, ketorolac, and or the like or any combination thereof.

The terms “distal” or “proximal” are used in the following description with respect to a position or direction relative to a reference point (e.g., such as a user [e.g., the treating physician or medical interventionalist]). “Distal” or “distally” are a position distant from or in a direction away from the reference point. “Proximal” or “proximally” or “proximate” are a position near or in a direction toward the reference point.

An increasing amount of care and resources has been focused on creating effective treatments for pain and infection care. The most common method of treating pain and infection includes oral and intravenous (IV) administration of drugs. Although these methods are prevalent, oftentimes the efficacy of the drug delivery method is lacking. For example, when drugs are administered orally or via IV methods, their treatment effect is generalized and not localized—the drug targets the entire patient instead of a localized treatment site. To overcome some of these issues, delivery vehicles such as topical treatments and, more particularly, hydrogel compounds have been developed to provide spatial and temporal control over the release of various therapeutic agents, including small molecule drugs, peptides, and cells. However, these treatments can also have drawbacks. These often synthetic carriers of therapeutics can be costly and, since they are fragile polymer chains, it can be difficult to sterilize polymer chains of the solution/hydrogel combination.

More recently, the concept of electrospraying treatment solutions, including antibacterial and/or analgesic solutions, to treatment sites has been considered. Electrostatic spraying is a technique that subjects a treatment solution to an electric field to charge the fluid. The electric field, provided by a voltage source, can create a charge to the fluid being administered (e.g., a positive charge or a negative charge). This is particularly helpful in the biomedical arena since the natural resting state of human cells is negative (i.e., a state of negative charge). This imbalance is created by potassium and sodium ions inside and outside the cell that establishes electrical capacity in a patient's body. This polarity differential creates a natural attraction between the treatment site and the solution being sprayed.

However, the negative/positive attraction is not the only benefit of electrostatic spraying. For example, subjecting the fluid to this electrical field can also produce diminutive droplets (e.g., micron sizes), providing a relatively uniformly distributed layer of treatment solution. When the electrical stresses due to the charge builds in a liquid droplet beyond its surface tension, the droplet disintegrates and/or atomizes into very fine droplets-which is known as Rayleigh disintegration or coulomb fission. As discussed herein, the term “atomize” is understood as some or all of the process of converting a substantially liquid solution into very fine particles or droplets. The solvent dielectric constant or conductivity can play a crucial role in dictating particulate morphology. Other factors that affect the way a liquid atomizes include vapor pressure, viscosity and miscibility of the treatment solution, voltage applied to the solution, etc.

It is noted that prior designs for electrosprayer devices did not take these various types of parameters into consideration. This is because most prior art electrostatic device focused on spraying one type of solution-consider the common examples of ink jet sprayers, paint sprayers, etc., which sprayed consistent solutions at consistent flow rates and with consistent voltage potentials. Further, these types of applications did not focus on operating parameters. The present disclosure provides solutions that can maintain sterility for each administration of a treatment solution by providing individualized, pre-filled disposable cartridges housing the components used for electrospraying. Further, each disposable cartridge can be individually tailored and/or communicate with a reusable electrostatic applicator to individually tailor the parameters needed to administer the preferred particle (e.g., nano to microparticles) droplets for targeted treatment.

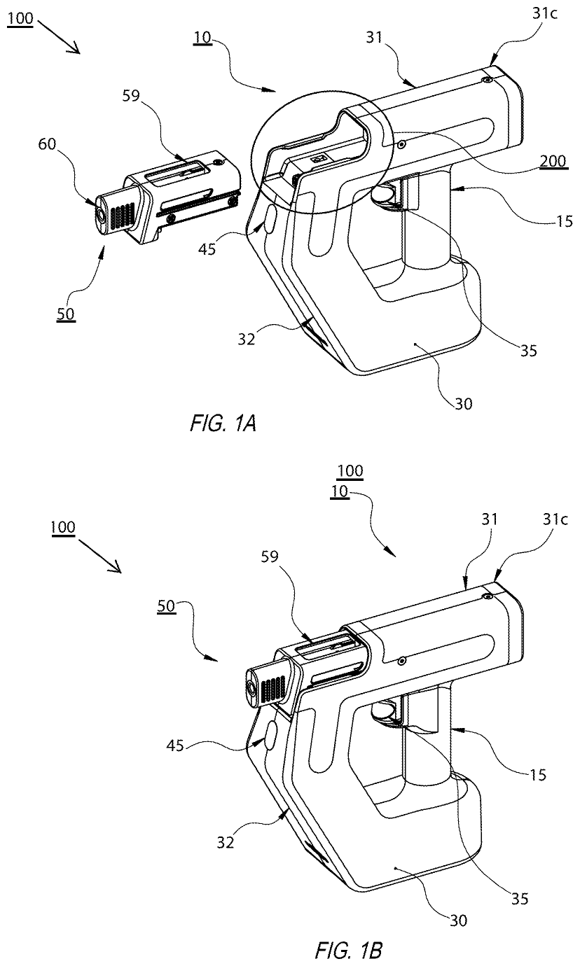

Turning to the drawings, FIG. 1A provides an example applicator 100 positioned in an exploded state with a disposable cartridge 50 while FIG. 1B depicts a perspective view of the applicator 100 of FIG. 1A with the cartridge 50 in an assembled state. Applicator 100 can include an applicator housing 10 which can include an upper portion 31, a lower base portion 30 at the lower section, and a handle portion 15 between portions 30 and 31. A forward cartridge support portion 32 can also be positioned between portions 30 and 31. In some aspects, an obtuse angle can be formed between aspects of outer surfaces of portions 30 and 32 while handle portion 15 can be orthogonal to portions 30 and/or 31. And while shown as a handheld pistol shaped device, nothing requires the reusable applicator 100 to have a pistol-shaped design, as the components herein can also be combined in other electrosprayer designs, for example and not limitation a fully-cylindrical, handheld electrosprayer design.

In some aspects, portion 30 can house a rechargeable battery that provides the voltage potential to create the electrical field at the nozzle assembly 60 of cartridge 50 and/or can power the components of applicator 100 (e.g., CPU and/or a high-voltage module disposed within the housing 10). The rechargeable battery can include one or more batteries, including for example direct current batteries such as lithium ion batteries, solid state batteries, etc. The rechargeable battery can provide sufficient voltage to create the voltage potential described above, including but not limited to approximately 1 V to approximately 40 kV. In some examples, the range can be approximately 1 V to 8 kV. In some examples, the voltage supply of the rechargeable battery can be one or more rechargeable batteries. In some examples, applicator 100 can include an actuator, e.g., a button 35 that can activate and/or initiate the components of applicator 100. For example, the button 35 can initiate power from the rechargeable battery and by activating and/or initiating power, the system is powered to electrostatically charge the liquid solution of the cartridge 50 via direct charging, induction charging, indirect charging, or any combinations thereof. In the case of direct charging, liquid solution of the cartridge 50 can flow through an electrically conductive tube or other conduit that is electrostatically charged such that the liquid solution is contacted and charged by direct contact.

In some examples, battery B can power the high voltage (HV) module 86, air pump 83, circuit board 64, target sensor 45, motor 90, user interface 87, one or more processors of applicator 100 (e.g., central processing unit (CPU)) as well as components of cartridge 50. As will be described in greater detail below, other features can be included in the applicator 100 that can obviate the need for button 35, including for example accelerometers, activation inputs from a user device, etc.

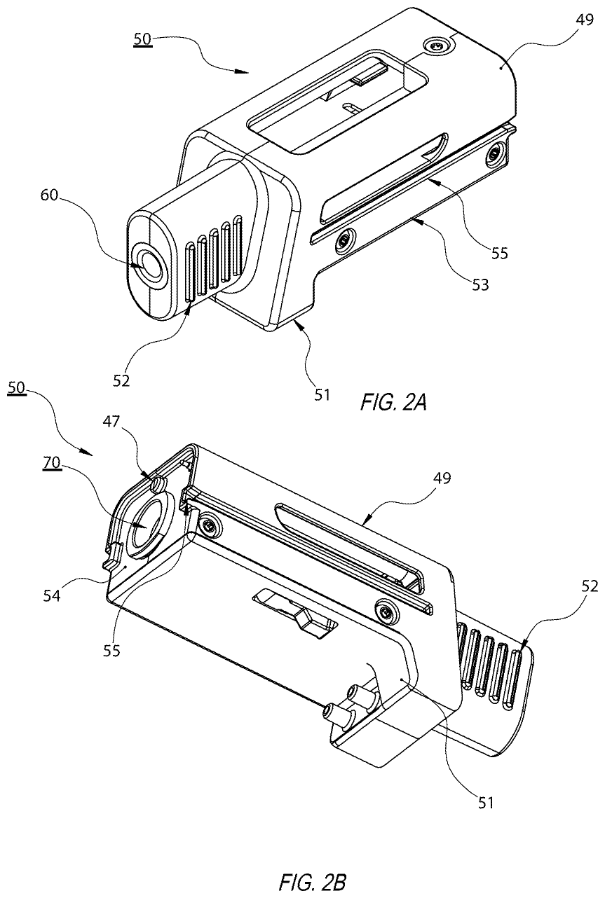

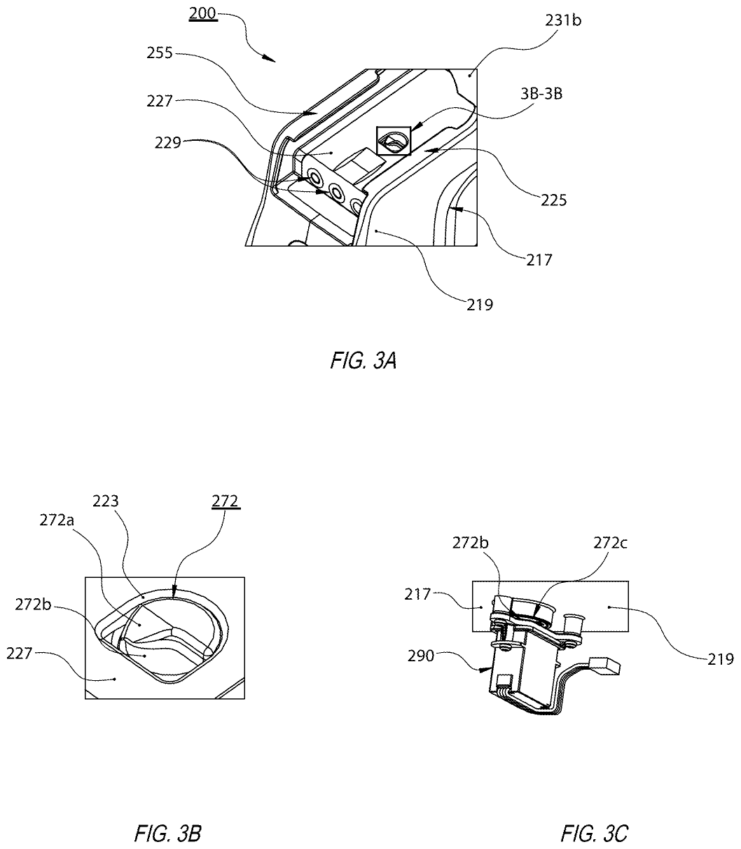

Applicator housing 10 can include a cartridge receiving chamber 200 more particularly shown in FIG. 3A. The cartridge receiving chamber 200 can be sized and positioned to accept the housing 49 of the disposable cartridge 50. Referring back to FIG. 2A, a forward perspective view of cartridge 50 is provided while FIG. 2B depicts a rear perspective view of cartridge 50. It is understood that in some aspects, the entire nozzle housing 60 of cartridge 50 can be molded (e.g., insert molded) as a single, integrally formed part and/or formed of multiple parts or sections. The nozzle housing 60 can include a nozzle outlet channel on a distal end that can be funnel shaped, sized and positioned to contain the outlet end of delivery tube. The nozzle housing 60 can include an air inlet port in fluid communication with the nozzle end of the air supply tube and the inlet port of cartridge 50. The nozzle housing 60 can include a voltage cavity port for connecting to a voltage wire. One side of the port can include a contact section through which the wire can pass to contact delivery tube. The nozzle outlet channel of housing 60 can be positioned at a distal end thereof and can receive air and fluid to expel fluid droplets that are charged by the voltage wire. In some aspects, the voltage wire can electrostatically charge contents within the delivery tube of cartridge 50 as well as fluid contents proximal thereof (e.g., contents within a barrel portion of the syringe 70).

Housing 49 can be formed of a multi-part shell with an aligning groove 55 that can engage with the aligning tab 225 of cartridge receiving chamber 200. Housing 49 can be formed and/or assembled in a number of ways, including but not limited to machining, molding, injection molding, three-dimensional printing, or any other suitable manufacturing process. Suitable materials for housing 49 may include one or more of glass filled nylon, glass filled polypropylene, glass filled polyethylene, polypropylene, polyethylene, or a plastic material. In some examples, housing 49 can include two or more sections of moldable plastic, e.g., a first half and a second half. The sections of housing 49 can be assembled together with fasteners (e.g., screws, rivets, a weld [e.g., a sonic weld], one or more straps or snaps, an adhesive or adhesive tape, etc.) such that the internal components are disposed between the portions and/or respective halves of housing 49. In some aspects, housing 49 can include a housing spray outlet associated with nozzle assembly 60 that enables charged treatment solution to be expelled from cartridge 50.

Groove 55 can engage with tab 225 to align housing into the cartridge receiving chamber 200 to facilitate proper alignment between cartridge 50 and chamber 200. Housing 49 can include grooves 55 positioned on opposite lateral sides of cartridge 50. Groove 55 can include an open proximal end and a distal end. In some aspects, the distal end can be closed. In some aspects, the rear of cartridge 50 can include one or more stopping surfaces to prevent further sliding of cartridge 50 with respect to tab 225. In those aspects where the distal end of groove 55 is closed and adjacent or otherwise towards the nozzle housing 60, the distal end prevents tab 225 from sliding deeper into groove 55.

FIG. 2B specifically shows the supply end and contact end of cartridge 50. An HV contact can be a contact port embedded on HV wall 54 and/or otherwise positioned on a face of cartridge HV wall 54, which is configured to mount against an HV wall (not shown but adjacent the cartridge receiving chamber 200, as shown and described in U.S. application Ser. No. 18/110,854, the contents of which are incorporated herein by reference as if set forth verbatim. HV wall 54 can also include an air intake port 47 which can be configured to couple to a corresponding air intake port of the HV wall.

As will be further discussed, cartridge 50 can be securely engaged with and/or released from chamber 200 in a number of approaches. In FIGS. 3A to 3C, an exemplary system and approach is disclosed for securely engaging and/or releasing cartridge 50 from chamber 200. Specifically, FIG. 3A shows a perspective view of chamber 200 with a distal stepped portion through which connecting ports 229 can connect to aspects of cartridge 50 (e.g., air supply ports). Chamber 200 can include a lower forward portion 219 with an outer stepped portion 217 extended distally therefrom. Chamber 200 may also include an outer rim edge 231b defining an outermost perimeter of chamber 200.

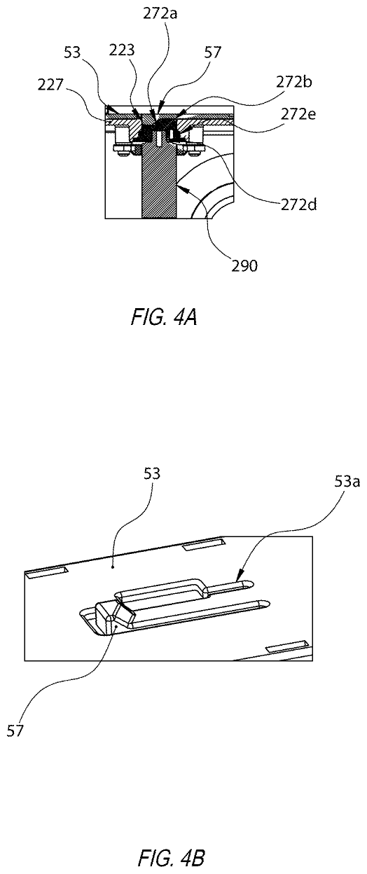

FIG. 3B shows a close-up of section 3B-3B of FIG. 3A, which illustrates aspects of a cartridge retention system configured to engage with and retain the cartridge housing 49 in a connected configuration with chamber 200. FIG. 3C further shows a lower internal perspective view of a motor 290 operatively connected to portion 217. Specifically, motor 290 (e.g., a servo motor) is configured to rotate a cam release 272 so as to urge and release (e.g., pop up) a latch 57 of the cartridge 50. The latch 57 is more particularly seen in FIG. 4B, with one or more perimetral cutouts 53a of surface 53. The cam release 272 can include an angled surface 272a that can include a catch, where the surface 272a can extend from a wider base portion 272b. Cam release 272 can be configured to extend through cutout 223 of surface 227. As shown more clearly in the cross-sectional view of FIG. 4A, cam release 272 can include a washer surface 272c configured to attach to surface with a shaft 272d extended away from surface 272c and surface 217. A seal 272e (e.g., a gasket, an O-ring, etc.) can be positioned between surface 272c and surface 227 of the housing 49. Motor 290 can be mounted to one or more mounts of cartridge receiving chamber 200 and be rotatably connected to shaft 272d. In this respect, motor 90 can be a servo motor configured to move and/or otherwise rotate surface 272c, portion 272b, and surface 272a.

An exemplary latch 57 is shown in the close-up lower-perspective view of FIG. 4B, which shows aspects of cartridge 50. Latch 57 can include an angled face projecting from a lower surface 53 of the cartridge housing 49 to securely connect with an angled surface 272a of the cam release 272 of the cartridge receiving chamber 200. It is understood that surface 53 is not so limited and can instead be a side surface or an upper surface of cartridge 50. Once the latch 57 is released, one or more bias members (e.g., springs, resilient materials, etc.) on the applicator side urge the cartridge 50 out of the chamber 200.

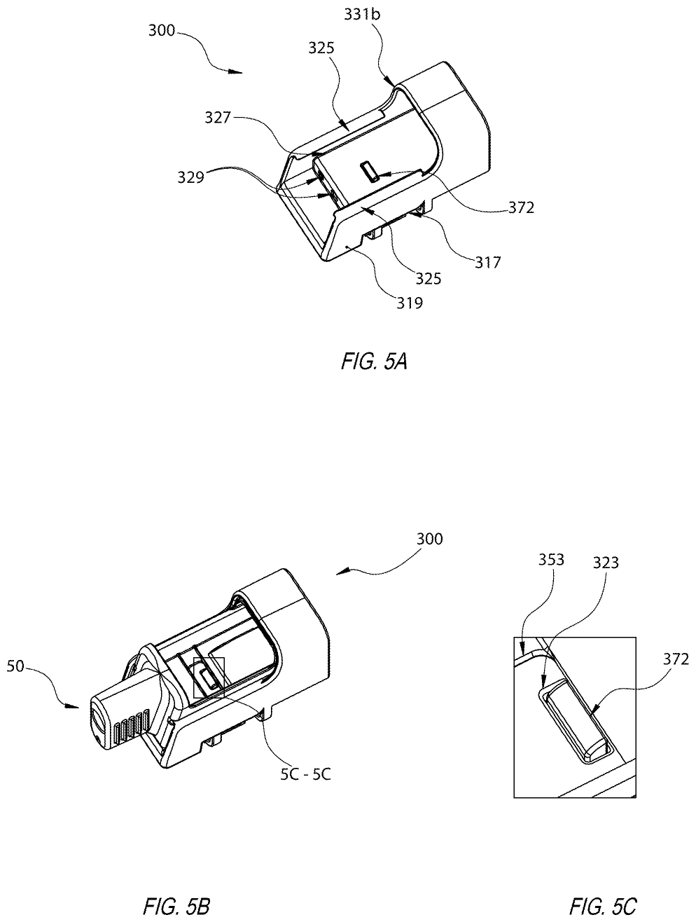

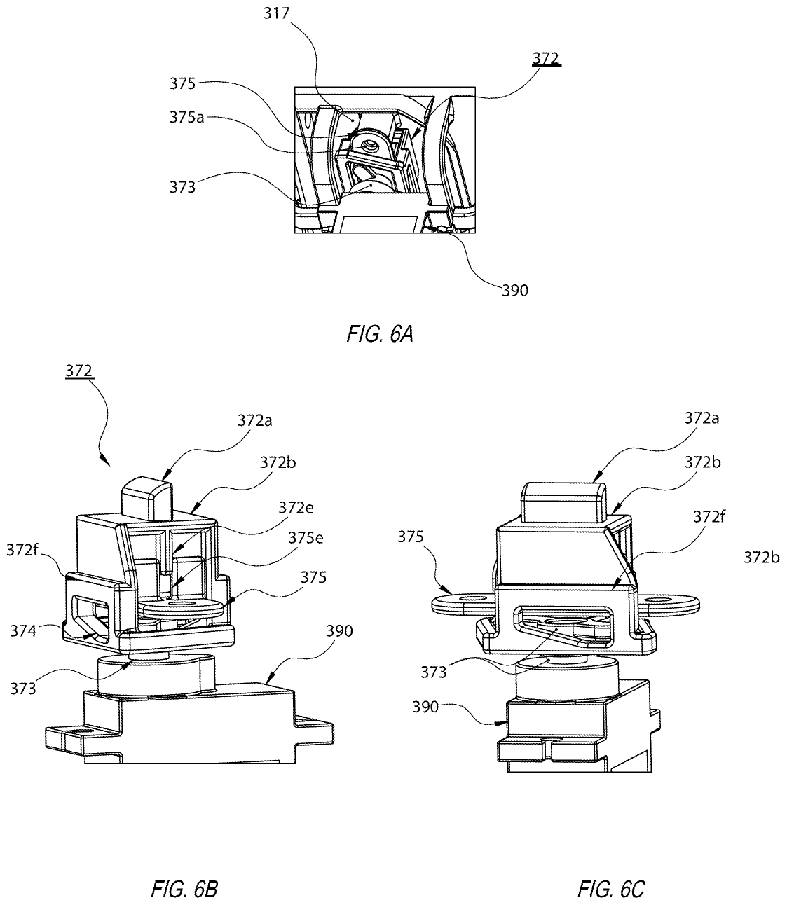

In FIGS. 5A to 6C, another exemplary system and approach is disclosed for securely engaging and/or releasing cartridge 50 from another exemplary cartridge receiving chamber 300. FIG. 5A shows a perspective view of cartridge receiving chamber 300 while FIG. 5B shows a perspective view of cartridge 50 when coupled with chamber 300 according to exemplary aspects of this disclosure. Specifically, FIG. 5A shows a perspective view of chamber 300 with a distal stepped portion through which connecting ports 329 can connect to aspects of cartridge 50 (e.g., air supply ports). Chamber 300 can include a lower forward portion 319 with an outer stepped portion 317 extended distally therefrom. Chamber 300 may also include an outer rim edge 331b defining an outermost perimeter of chamber 300.

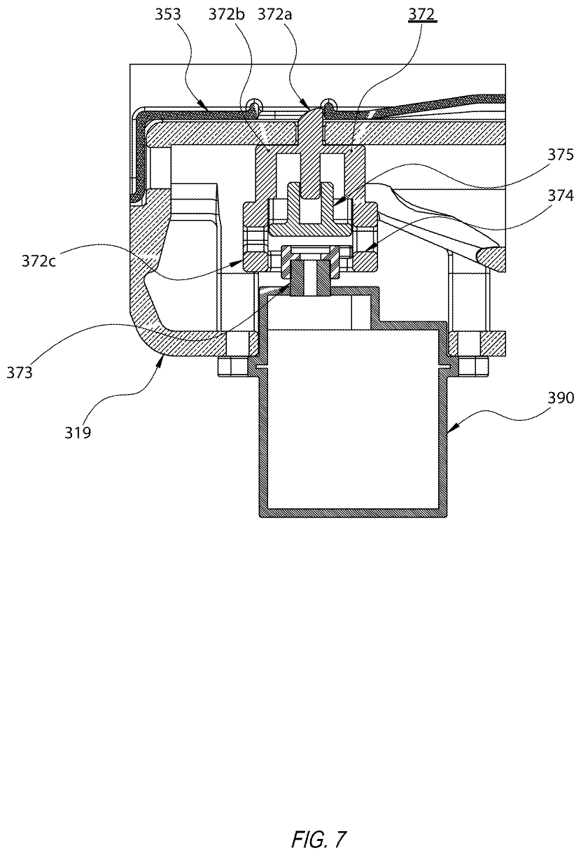

FIG. 5C shows a close-up of section 5C-5C of FIG. 5B, which illustrates aspects of another cartridge retention system configured to engage with and retain the cartridge housing 49 in a connected configuration with chamber 300. Specifically, it can be seen that a wall (e.g., the lower wall) of the cartridge housing 49 includes a larger recessed area 353 with a relief 323. The relief 323 can be an aperture or cutout selectively positioned to align with a corresponding pull-down lock 372. FIG. 6A further shows a lower internal perspective view of a motor 390 operatively connected to lock 372 and port 317 of chamber 300. FIGS. 6B to 6C show perspective views of aspects of the motor 390 when coupled to the lock 372. FIG. 7 shows a side cross-section view of aspects of the exemplary motor 390 operatively connected to aspects shown previously in FIGS. 5A to 6C. Specifically, motor 390 (e.g., a servo motor) is configured to rotate on a ramped face 374 of a pull-down lock 372. Lock 372 can include a housing and/or be substantially hollowed-out and include a lower base portion 372c that includes a wider footprint than the rest of lock 372. A plurality of lateral protrusions 372f can extend away from portion 372c whereby the ramped face 374 can be formed in each of the lateral protrusions 372f. Face 374 can be a multi-sided cutout or window with at least one inclined or otherwise ramped edge configured to receive aspects of a planar catch 373. An intermediate portion 372b can extend from the lateral protrusions 372f of faces 374. An upper catch 372a can extend from portion 372b. Catch 372a can include a smaller footprint than that the rest of portions 372c and 372. Catch 372a can include an angled outermost edge including a ramped catch to releasably engage with a corresponding receiver of the housing 49. A guide 372e can extend from at least partially from aspects of portion 372b towards base portion 372c.

Motor 390 can be rotatably connected to a planar catch 373. Catch 373 can include a lower portion configured to rotatably couple and be rotated by the motor 390. Catch 373 can include an upper wider top portion configured so that rotating along face 374 causes catch 373 to be extended deeper into a cavity within lock 372. In turn, a restriction guide 375 can include a lower base portion with one or more outer apertures 375a extended through the lower base for mounting to corresponding connectors associated with chamber 300. One or more guides 375e can extend upwardly therefrom with an open ended groove to slidably engage with guide 372e. Once the one or more guides 375e are slidably coupled with guide 372e, guide 375 can keep the lock 372 moving only in a vertical and/or angled direction (e.g., up and down motion), approximately vertical direction (e.g., vertical direction with a possible variation of ±10%), as well as approximately +/−45° in the process of transitioning between connected and disconnected configurations.

In some aspects, lock 372 is backed by one or more bias elements (e.g., a spring, a material with resistance such as an elastomer, torsional member, coil, etc.) that maintain, by the upward urging force of the one or more bias elements, corresponding catch 372a in an upright position within chamber 300. In turn, when the cartridge housing 49 slides over catch 372a, the cartridge 50 is locked in place when the relief 323 in the cartridge aligns catch 372a of the lock 372.

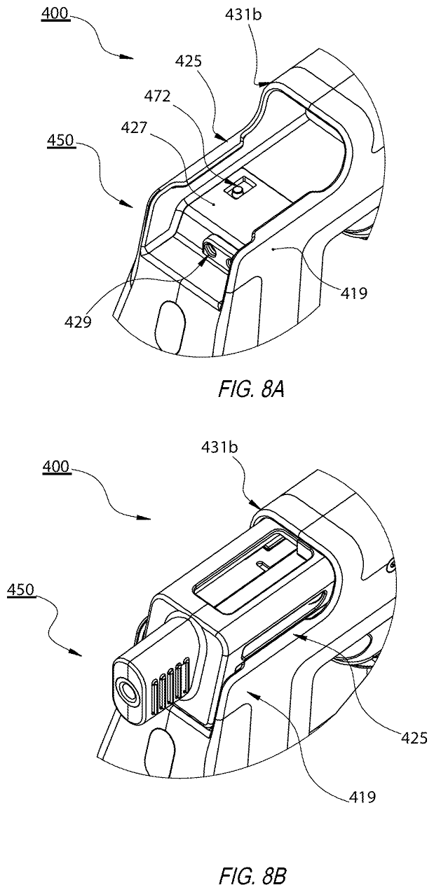

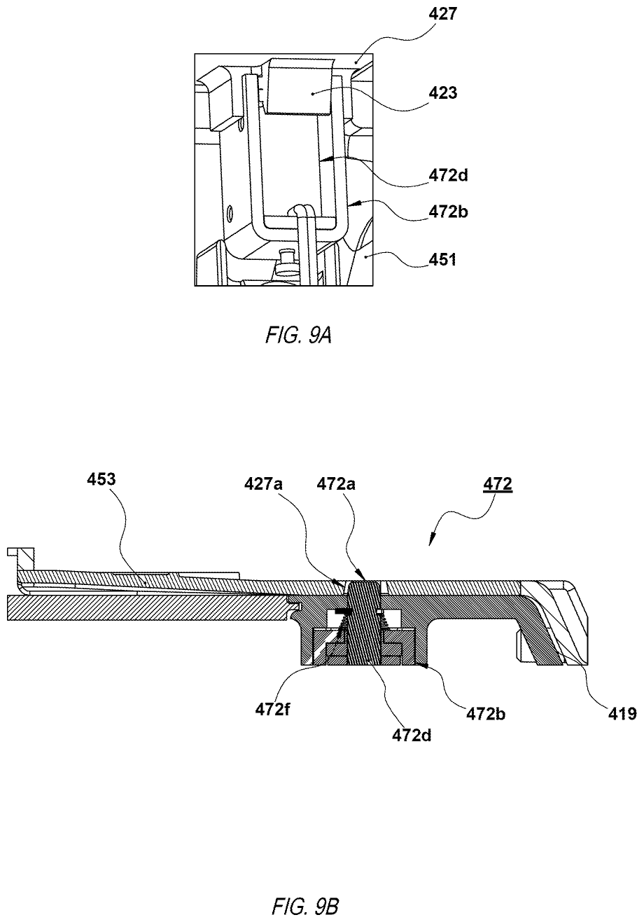

In FIGS. 8A to 9B, another exemplary system and approach is disclosed for securely engaging and/or releasing cartridge 50 from another exemplary cartridge receiving chamber 400. FIG. 8A shows a perspective view of cartridge receiving chamber 400 while FIG. 8B shows a perspective view of cartridge 50 when coupled with chamber 400 according to exemplary aspects of this disclosure. Specifically, FIG. 8A shows a perspective view of chamber 400 with a distal stepped portion through which connecting ports 429 can connect to aspects of cartridge 50 (e.g., air supply ports). Chamber 400 can include a lower forward portion 419 with forward internal portion 427 through which a solenoid pin 472 can be extended. Chamber 400 may also include an outer rim edge 431b defining an outermost perimeter of chamber 400.

FIG. 9A shows a lower internal perspective view of a lower surface of the cartridge receiving chamber 400 while FIG. 9A shows a lower internal perspective view of a lower side of surface 427. In operation of chamber 400, the cartridge 50 slides over solenoid pin 472 (e.g., a pull solenoid). Pin 472 in turn is caused to be pushed down by the cartridge 50 as it slides over, then allows the solenoid pin 472 to pop up through a cutout 427a in a lower surface of housing 49 thereby locking the cartridge 50 in place. When the cartridge 50 is to be ejected from chamber 400, the solenoid pin 472 is energized and the pin 472 gets pulled down, allowing the cartridge 50 to be removed from chamber 400. As shown in FIGS. 9A and 9B, the solenoid pin can include an uppermost end 472a configured to pass through the cutout 427a. One or more bias elements 472f (e.g., a spring, a material with resistance such as an elastomer, torsional member, coil, etc.) can be configured to support pin 472 and cause pin 472 to transition between the disconnected configuration to popping back in place in the connected configuration. Pin 472 can further include an elongate portion extended from end 472a back into pin housing 472b. The one or more bias elements 472f can be positioned between the end 472a and the pin housing 472b. Pin housing 472b can be completely enclosed or partially enclosed as shown in FIG. 9A, where at least one lateral face is absent. As shown in FIG. 9B, cartridge 50 can include a ramp 453 to facilitate urging down the solenoid pin 472 during cartridge 50 loading into chamber 400.

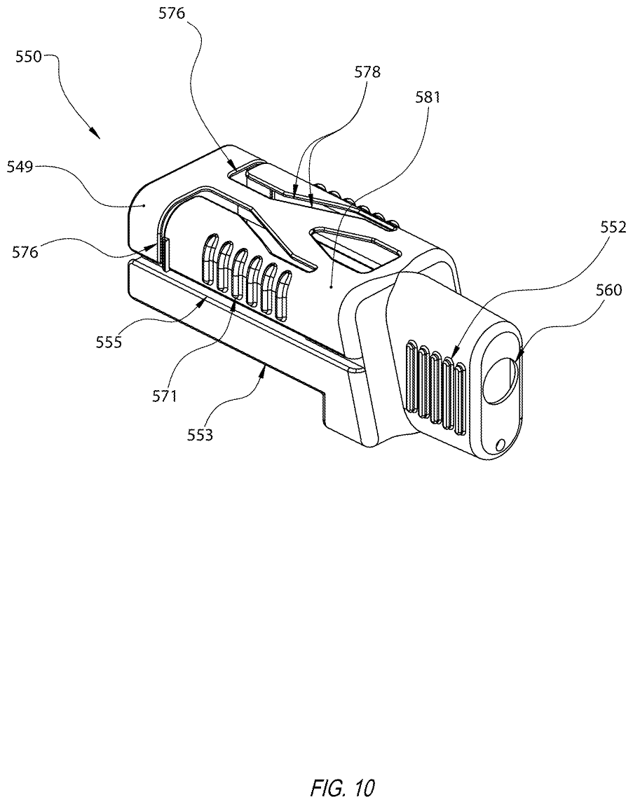

FIG. 10 shows a perspective view of an example cartridge 550 according to exemplary aspects of this disclosure. Similar to cartridge 50, cartridge 550 can include nozzle assembly 560 as well as a cartridge housing 549 formed of a multi-part shell with an aligning groove 555 that can engage with an aligning tab (e.g., tab 225 of cartridge receiving chamber 200). In some examples, one or more snap locks 576 can be positioned with groove 555 and be extended outward from an outer surface of housing 549. One or more reliefs 578 can extend upward from respective groove 555 along locks 576 and pass by upper edge of housing 549. An outer surface of housing 549 can also include one or more grip inducing members 571 (e.g., one or more outwardly extended protrusions from the outer surface of housing 549) and one or more living hinges 581. In operation, members 571 can be compressed by a user causing the cartridge 550 to snap into recesses of a corresponding cartridge receiving chamber and allow the cartridge 550 to lock in place. In some aspects, a user would can manually press in members 571 to allow, by the hinge 581 formed therewith, the cartridge 550 to be removed from the cartridge receiving chamber.

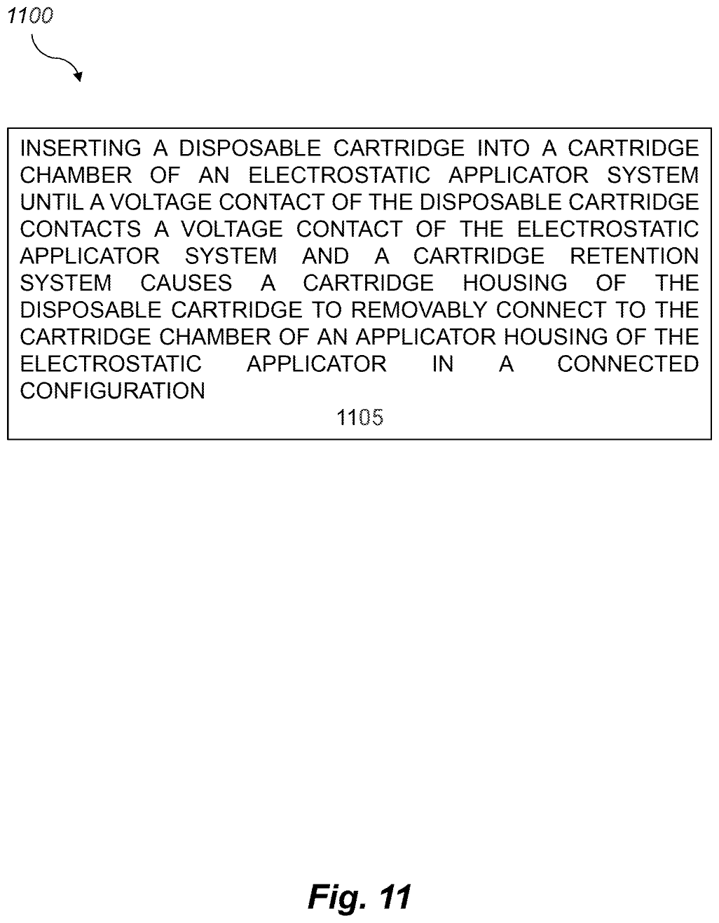

FIG. 11 is a flow chart of an exemplary method 1100 for operating an electrostatic applicator system. In method 1100, step 1105 can include inserting a disposable cartridge into a cartridge chamber of an electrostatic applicator system until a voltage contact of the disposable cartridge contacts a voltage contact of the electrostatic applicator system and a cartridge retention system causes a cartridge housing of the disposable cartridge to removably connect to the cartridge chamber of an applicator housing of the electrostatic applicator in a connected configuration.

In some aspects, the method 1100 can include rotating, by a motor within a device housing of the electrostatic applicator, a cam release thereby releasing a latch of the cartridge retention system so that the cartridge housing transitions to a disconnected configuration.

In some aspects, the method 1100 can include upon releasing the latch, one or more bias elements of the cartridge chamber cause the cartridge housing to be urged away from the cartridge chamber.

In some aspects, the method 1100 can include engaging a relief of the cartridge retention system with a pull-down lock extended through an aperture in the cartridge chamber from within the device housing, the pull-down lock comprising a ramped face; and rotating, by a motor within the device housing, a restriction guide on and/or along a ramped face of the pull-down lock so that the motor is only capable of moving, by the restriction guide, in a vertical and/or angle direction.

In some aspects, the method 1100 can include releasing the cartridge housing from the connected configuration to a disconnected configuration by squeezing one or more compressible portions of the cartridge housing that form a living hinge so as to unlock the cartridge housing from the cartridge chamber.

In some aspects, the method 1100 can include causing, by a lower surface of the cartridge housing, a pin of a solenoid extended from within the device housing through an aperture of a wall of the cartridge chamber, to be pushed inward as the cartridge housing slides within the cartridge chamber until the pin is released to extend through an aperture in the cartridge retention system thereby locking the cartridge housing in the connected configuration.

In some aspects, the method 1100 can include transitioning the cartridge housing to a disconnected configuration by energizing the solenoid so that the pin is released from the aperture in the outer surface of the cartridge housing.

Although systems and methods have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-11 may be modified, and that the foregoing discussion of certain of these embodiments does not necessarily represent a complete description of all possible embodiments. For example, one or more of the procedures, processes, or activities of FIG. 11 may include different procedures, processes, and/or activities and be performed by some different modules, in some different orders.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. A disposable fluid delivery system for an electrostatic applicator, comprising:
a cartridge housing configured to store an aqueous solution;
a nozzle housing;
a high voltage contact electrically attached to a delivery tube at least partially within the nozzle housing, the high voltage contact configured to be in electrical communication with a high voltage module and electrostatically charge fluid contents within the delivery tube; and
a cartridge retention system positioned at least partially with an outer surface of the cartridge housing, the cartridge retention system configured to engage with and retain the cartridge housing in a connected configuration with a cartridge receiving chamber of an applicator housing of the electrostatic applicator.

2. The disposable fluid delivery system of claim 1, the cartridge retention system comprising a latch comprising an angled face projecting from a surface of the cartridge housing and configured to securely connect with an angled surface of a cam release of the cartridge receiving chamber.

3. The disposable fluid delivery system of claim 1, the cartridge retention system comprising a pull-down lock comprising a ramped face; and
a restriction guide slidably connected to the pull-down lock.

4. The disposable fluid delivery system of claim 3, wherein the pull-down lock comprises a lock housing with an upper ramped catch, and wherein the ramped face is positioned on an outer edge of the lock housing adjacent or on a surface opposite the upper ramped catch.

5. The disposable fluid delivery system of claim 3, wherein the restriction guide comprises a planar base with a guide of the restriction guide extended upward away from the planar base, wherein the restriction guide is connected to a spring.

6. The disposable fluid delivery system of claim 1, the cartridge retention system comprising one or more compressible portions of the cartridge housing that form a living hinge configured to snap into recesses.

7. The disposable fluid delivery system of claim 1, the cartridge retention system comprising an aperture in the outer surface of the cartridge housing, the aperture arranged to receive a movable pin of a solenoid that locks the cartridge housing in a connected configuration.

8. An electrostatic applicator system for delivering a treatment solution to a target site, comprising:
a portable reusable electrostatic applicator comprising a device housing configured to be handheld, a motor in the device, a voltage source, a high voltage module electrically connected to the voltage source, and a cartridge chamber defined with one or more outer surfaces of the device housing; and
a disposable cartridge removably insertable in the cartridge chamber, the disposable cartridge comprising:
a cartridge housing configured to an aqueous solution;
a nozzle housing;
a high voltage contact electrically attached to a delivery tube at least partially within the nozzle housing, the high voltage contact configured to be in electrical communication with a high voltage module and electrostatically charge fluid contents within the delivery tube;
a cartridge retention system positioned at least partially with an outer surface of the cartridge housing, the cartridge retention system configured to engage with and retain the cartridge housing in a connected configuration with a cartridge receiving chamber of an applicator housing of the electrostatic applicator

9. The electrostatic applicator system of claim 8, further comprising:
a cam release extended from within the device housing through an aperture of a wall of the cartridge chamber, wherein a motor within the device housing is configured to rotate the cam release and release a latch of the cartridge retention system and cause the cartridge housing to transition to a disconnected configuration.

10. The electrostatic applicator system of claim 9, further comprising one or more bias elements of the cartridge chamber, wherein upon releasing the latch, the one or more bias elements cause the cartridge housing to be urged away from the cartridge chamber.

11. The electrostatic applicator system of claim 9, wherein the latch of the cartridge retention system is angled to receive an angled face of the cam release.

12. The electrostatic applicator system of claim 8, further comprising:
a pull-down lock configured to engage with a relief of the cartridge retention system, the pull-down lock extended from within the device housing through an aperture of a wall of the cartridge chamber, the pull-down lock comprising a ramped face; and
a restriction guide slidably connected to the pull-down lock, wherein a motor within the device housing is configured to rotate on the ramped face of the pull-down lock so that the motor is only capable of moving, by the restriction guide, in a vertical and/or angled direction.

13. The electrostatic applicator system of claim 12, wherein the pull-down lock comprises a lock housing with an upper ramped catch extended through the aperture, and wherein the ramped face is positioned on an outer edge of the lock housing adjacent or on a surface opposite the upper ramped catch.

14. The electrostatic applicator system of claim 12, wherein the restriction guide comprises a planar base with a vertical guide of the restriction guide extended upward away from the planar base, wherein the restriction guide is connected to a spring that maintains the pull-down lock when the cartridge housing slides into the cartridge chamber thereby locking the cartridge housing in place when the relief in the cartridge housing aligns with the pull-down lock.

15. The electrostatic applicator system of claim 8, wherein the cartridge retention system comprises one or more compressible portions of the cartridge housing that form a living hinge configured to snap into recesses of the cartridge chamber so as to lock the cartridge housing in the cartridge chamber.

16. The electrostatic applicator system of claim 8, further comprising:
a solenoid comprising a pin extended from within the device housing through an aperture of a wall of the cartridge chamber, wherein the pin is caused to be pushed inward as the cartridge housing slides within the cartridge chamber over the pin of the solenoid until the pin is released to extend through an aperture in the cartridge retention system thereby locking the cartridge housing in a connected configuration.

17. The electrostatic applicator system of claim 16, wherein to transition the cartridge housing to a disconnected configuration, the solenoid is energized and the pin is released from the aperture in the outer surface of the cartridge housing.

18. A method for operating an electrostatic applicator system, comprising:
inserting a disposable cartridge into a cartridge chamber of an electrostatic applicator system until a voltage contact of the disposable cartridge contacts a voltage contact of the electrostatic applicator system and a cartridge retention system causes a cartridge housing of the disposable cartridge to removably connect to the cartridge chamber of an applicator housing of the electrostatic applicator in a connected configuration.

19. The method of claim 18, further comprising:
rotating, by a motor within a device housing of the electrostatic applicator, a cam release thereby releasing a latch of the cartridge retention system so that the cartridge housing transitions to a disconnected configuration.

20. The method of claim 19, further comprising:
upon releasing the latch, one or more bias elements of the cartridge chamber cause the cartridge housing to be urged away from the cartridge chamber.
